(19) United States
(12) Patent Application Publication
(10) Pub. No.: US 2014/0336482 A1
(43) Pub. Date: Nov. 13, 2014

(54) LASER DEVICE AND PHOTOACOUSTIC MEASUREMENT DEVICE

(71) Applicant: FUJIFILM Corporation, Tokyo (JP)

(72) Inventors: Tadashi KASAMATSU, Ashigarakami-gun (JP); Kazuhiro HIROTA, Ashigarakami-gun (JP)

(21) Appl. No.: 14/337,761

(22) Filed: Jul. 22, 2014

Related U.S. Application Data
(63) Continuation of application No. PCT/JP2013/053385, filed on Feb. 13, 2013.

(30) Foreign Application Priority Data
Mar. 9, 2012 (JP) 2012-052498
Sep. 20, 2012 (JP) 2012-206754

(51) Int. Cl. H01S 3/117; A61B 5/145; A61B 5/00

(52) U.S. Cl. CPC H01S 3/117; A61B 5/0095; A61B 5/14546
USPC 600/322; 372/13

(57) ABSTRACT

Disclosed is a laser device which can emit light having first and second wavelengths, having the advantage of increasing laser efficiency without causing an increase in cost. A flash lamp irradiates excitation light onto a laser rod. An optical resonator includes a pair of mirrors facing each other with the laser rod interposed therebetween. A wavelength switching unit includes a long path filter which transmits light having a wavelength equal to or greater than a first wavelength. The wavelength switching unit inserts the long path filter on the optical path of the optical resonator when the wavelength of laser light to be emitted is the first wavelength.

### CROSS-REFERENCE TO RELATED APPLICATIONS

This application is a Continuation of PCT International Application No. PCT/JP2013/053385 filed on Feb. 13, 2013, which claims priority under 35 U.S.C §119(a) to Japanese Patent Application No. 2012-052498 filed Mar. 9, 2012 and Japanese Patent Application No. 2012-206754 filed Sep. 20, 2012. Each of the above application(s) is hereby expressly incorporated by reference, in its entirety, into the present application.

### BACKGROUND OF THE INVENTION

1. Field of the Invention

The present invention relates to a laser device, and in particular, a laser device which can emit light having first and second wavelengths. The present invention also relates to a photoacoustic measurement device including the laser device.

2. Description of the Related Art

In the related art, for example, as shown in JP2005-21380A or A High-Speed Photoacoustic Tomography System based on a Commercial Ultrasound and a Custom Transducer Array, Xueding Wang, Jonathan Cannata, Derek DeBusschere, Changhong Hu, J. Brian Fowlkes, and Paul Carson, Proc. SPIE Vol. 7564, 756424 (Feb. 23, 2010), a photoacoustic imaging device which images the inside of a living body using a photoacoustic effect is known. In this photoacoustic imaging device, for example, pulse light, such as pulse laser light, is irradiated onto the living body. Inside the living body onto which pulse light is irradiated, a tissue of the living body which absorbs energy of pulse light expands in volume due to heat, and an acoustic wave is generated. The acoustic wave is detected by an ultrasound probe or the like, and the inside of the living body can be visualized based on the detected signal (photoacoustic signal). In a photoacoustic imaging method, since an acoustic wave is generated in a specific optical absorber, a specific tissue in the living body, for example, a blood vessel or the like can be imaged.

On the other hand, a large number of living body tissues have an optical absorption characteristic which changes depending on the wavelength of light, and in general, the optical absorption characteristic is peculiar to each tissue. For example, FIG. 17 shows molecular absorption coefficients for each light wavelength of oxygenated hemoglobin (hemoglobin bonded to oxygen: oxy-Hb) contained in a large amount in a human artery and deoxygenated hemoglobin (hemoglobin not bonded to oxygen: deoxy-Hb) contained in a large amount in a vein. The optical absorption characteristic of the artery corresponds to the optical absorption characteristic of oxygenated hemoglobin, and the optical absorption characteristic of the vein corresponds to the optical absorption characteristic of deoxygenated hemoglobin. A photoacoustic imaging method which irradiates light having two different wavelengths inside a blood vessel portion by means of a difference in light absorptance depending on wavelength and images an artery and a vein distinctively is known (for example, see JP2010-046215A).

In regard to a variable wavelength laser, JP2009-231483A describes a laser in which an etalon filter or a birefringent filter as a wavelength selection element is disposed in an optical resonator. Laser light having a desired wavelength can be obtained by adjusting the rotation angle of the birefringent filter or the like. JP1998-65260A (JP-H10-65260A) describes a multicolor solid-state laser device which can easily switch and output laser light having a plurality of wavelengths. In JP1998-65260A (JP-H10-65260A), a bandpass filter which selectively transmits only light having a specific peak wavelength is disposed on an optical path between a laser active medium and one of optical resonator mirrors. The bandpass filters are prepared for peak wavelengths to be selected, and any of the prepared bandpass filters is disposed on the optical path, whereby it is possible to switch and output laser light having a plurality of wavelengths.

### SUMMARY OF THE INVENTION

In the related art, as a filter which controls an oscillation wavelength in a laser, a birefringent filter (BRF) or a bandpass filter (BPF) is used. However, there is a problem in that the birefringent filter is made of quartz and expensive. In general, there is a problem in that the bandpass filter has low light transmittance, and thus decreases in output intensity of output laser light. In compensating for the decrease in output with the insertion of the bandpass filter, there is a problem in that the laser device increases in size.

The invention has been accomplished in consideration of the above-described situation, and an object of the invention is to provide a laser device which can emit light having first and second wavelengths at low cost with high laser efficiency.

Another object of the invention is to provide a photoacoustic measurement device including the laser device.

In order to attain the above-described object, the invention provides a laser device which emits light having a plurality of wavelengths including a first wavelength and a second wavelength having a shorter wavelength than the first wavelength, and the second wavelength has a laser gain coefficient higher than a laser gain coefficient at the first wavelength in a laser gain coefficient wavelength characteristic, the laser device including a laser medium, an excitation light source which irradiates excitation light onto the laser medium, an optical resonator which has a pair of mirrors facing each other with the laser medium interposed therebetween, and a wavelength switching unit which includes a first long path filter, which transmits light having a wavelength equal to or greater than the first wavelength, and inserts the first long path filter on the optical path of the optical resonator when the wavelength of laser light to be emitted is the first wavelength.

When the wavelength of laser light to be emitted is the second wavelength, the wavelength switching unit may transmit light having the first and second wavelengths. The wavelength switching unit may further include a second long path filter, which transmits light having a wavelength equal to or greater than the second wavelength, and may insert the second long path filter on the optical path of the optical resonator when the wavelength of laser light to be emitted is the second wavelength.

The wavelength switching unit may have a first region where the first long path filter is disposed and a second region where the second long path filter is disposed, and may be configured as a filter rotor which can alternately insert the first region and the second region on the optical path of the optical resonator with rotation displacement.

The laser gain coefficient at the second wavelength in the laser gain coefficient wavelength characteristic may be maximal, and the wavelength switching unit may further include an optical member, which transmits at least light having the second wavelength, and may insert the optical member on the optical path of the optical resonator when the wavelength of laser light to be emitted is the second wavelength.

The wavelength switching unit may have a first region where the first long path filter is disposed and a second region where the optical member is disposed, and may be configured as a filter rotor which can alternately insert the first region and the second region on the optical path of the optical resonator with the rotation displacement.

The laser gain coefficient at the second wavelength in the laser gain coefficient wavelength characteristic may be maximal, and the wavelength switching unit may remove the first long path filter from the optical path of the optical resonator when the wavelength of laser light to be emitted is the second wavelength.

A dimmer member which decreases the amount of transmission of at least light having the second wavelength may be inserted on the optical path of the optical resonator or the optical path of emitted light from the optical resonator when the wavelength of laser light to be emitted is the second wavelength. In this case, it is preferable that the light transmittance of the dimmer member is selected such that the light intensity of light having the first wavelength output from the laser device and the light intensity of light having the second wavelength are identical.

Alternatively, the reflectance of a laser output-side mirror of the pair of mirrors for light having the first wavelength may be higher than the reflectance for light having the second wavelength. In this case, it is preferable that the reflectance of the laser output-side mirror for light having the first wavelength and the reflectance of the laser output-side mirror for light having the second wavelength are selected such that the effective gain of the optical resonator for the first wavelength and the effective gain of the optical resonator for the second wavelength are identical.

The input energy of excitation light to the laser medium may be the same between when the wavelength of laser light to be emitted is the first wavelength and when the wavelength of laser light to be emitted is the second wavelength.

The laser device may further include a Q switch which is disposed on the optical path of the optical resonator.

The invention also provides a photoacoustic measurement device including the laser device which emits light having a plurality of wavelengths including a first wavelength and a second wavelength having a shorter wavelength than the first wavelength, and the second wavelength has a laser gain coefficient higher than a laser gain coefficient at the first wavelength in a laser gain coefficient wavelength characteristic, the laser device having a laser medium, an excitation light source which irradiates excitation light onto the laser medium, an optical resonator which includes a pair of mirrors facing each other with the laser medium interposed therebetween, and a wavelength switching unit which includes a first long path filter, which transmits light having a wavelength equal to or greater than the first wavelength, and inserts the first long path filter on the optical path of the optical resonator when the wavelength of laser light to be emitted is the first wavelength, a detection unit which detects a photoacoustic signal generated in a subject when laser light having the first wavelength and the second wavelength is irradiated onto the subject and generates first photoacoustic data and second photoacoustic data corresponding to the first wavelength and the second wavelength, and an intensity ratio extraction unit which extracts the magnitude relationship of relative signal intensity between the first photoacoustic data and the second photoacoustic data.

The photoacoustic measurement device may further include a photoacoustic image construction unit which generates a photoacoustic image based on the first photoacoustic data and the second photoacoustic data.

The photoacoustic measurement device may further include an intensity information extraction unit which generates intensity information representing signal intensity based on the first photoacoustic data and the second photoacoustic data, in which the photoacoustic image construction unit may determine the gradation value of each pixel of the photoacoustic image based on the intensity information and may determine the display color of each pixel based on the extracted magnitude relationship.

The photoacoustic measurement device may further include a complex number unit which generates complex data, in which one of the first photoacoustic data and the second photoacoustic data is a real part and the other data is an imaginary part, and a photoacoustic image reconstruction unit which generates a reconstructed image from the complex data by a Fourier transformation method, in which the intensity ratio extraction unit may extract phase information as the magnitude relationship from the reconstructed image, and the intensity information extraction unit may extract the intensity information from the reconstructed image.

The invention also provides a laser device which emits light having a plurality of wavelengths including a first wavelength and a second wavelength having a longer wavelength than the first wavelength, and the second wavelength has a laser gain coefficient higher than a laser gain coefficient at the first wavelength in a laser gain coefficient wavelength characteristic, the laser device including a laser medium, an excitation light source which irradiates excitation light onto the laser medium, an optical resonator which includes a pair of mirrors facing each other with the laser medium interposed therebetween, and a wavelength switching unit which includes a first short path filter, which transmits light having a wavelength equal to or less than the first wavelength, and inserts the first short path filter on the optical path of the optical resonator when the wavelength of laser light to be emitted is the first wavelength.

In the above-described case, the wavelength switching unit may further include a second short path filter, which transmits light having a wavelength equal to or less than the second wavelength, and may insert the second short path filter on the optical path of the optical resonator when the wavelength of laser light to be emitted is the second wavelength.

The laser device of the invention can emit light having the first wavelength and light having the second wavelength shorter than the first wavelength. The laser gain coefficient at the second wavelength is higher than the laser gain coefficient at the first wavelength. Conversely, the laser gain coefficient at the first wavelength is lower than the laser gain coefficient at the second wavelength. In the invention, the first long path filter which transmits light having a wavelength equal to or greater than the first wavelength is inserted on the optical path of the optical resonator at the time of the emission of light having the first wavelength. In general, the long path filter has light transmittance higher than a bandpass filter, thereby increasing laser efficiency. The long path filter is inexpensive compared to a birefringent filter made of quartz, thereby reducing costs.

### BRIEF DESCRIPTION OF THE DRAWINGS

FIG. 1 is a block diagram showing a photoacoustic measurement device according to a first embodiment of the invention.

FIG. 2 is a block diagram showing the configuration of a laser light source unit of the first embodiment.

FIGS. 3A and 3B are block diagrams showing the internal configuration of an optical resonator of the laser light source unit.

FIG. 4 is a graph showing a gain of alexandrite.

FIG. 5 is a graph showing light transmittance of a wavelength switching unit.

FIG. 6 is a graph showing an effective gain of the optical resonator.

FIG. 7 is a diagram showing a modification example of the wavelength switching unit.

FIG. 8 is a flowchart showing an operation procedure of the photoacoustic measurement device.

FIG. 9 is a diagram showing a configuration example of a wavelength switching unit in a laser device according to a second embodiment of the invention.

FIG. 10 is a graph showing the wavelength characteristics of light transmittance of first and second long path filters.

FIG. 11 is a graph showing an effective gain of an optical resonator.

FIG. 12 is a graph showing a wavelength characteristic of reflectance of an output mirror.

FIG. 13 is a block diagram showing the internal configuration of an optical resonator of a laser light source unit according to a modification example.

FIG. 14 is a block diagram showing a photoacoustic measurement device according to a third embodiment of the invention.

FIG. 15 is a graph showing light transmittance of a wavelength switching unit including a short path filter.

FIG. 16 is a graph showing an effective gain of an optical resonator.

FIG. 17 is a graph showing molecular absorption coefficients for each light wavelength of oxygenated hemoglobin and deoxygenated hemoglobin.

### DESCRIPTION OF THE PREFERRED EMBODIMENTS

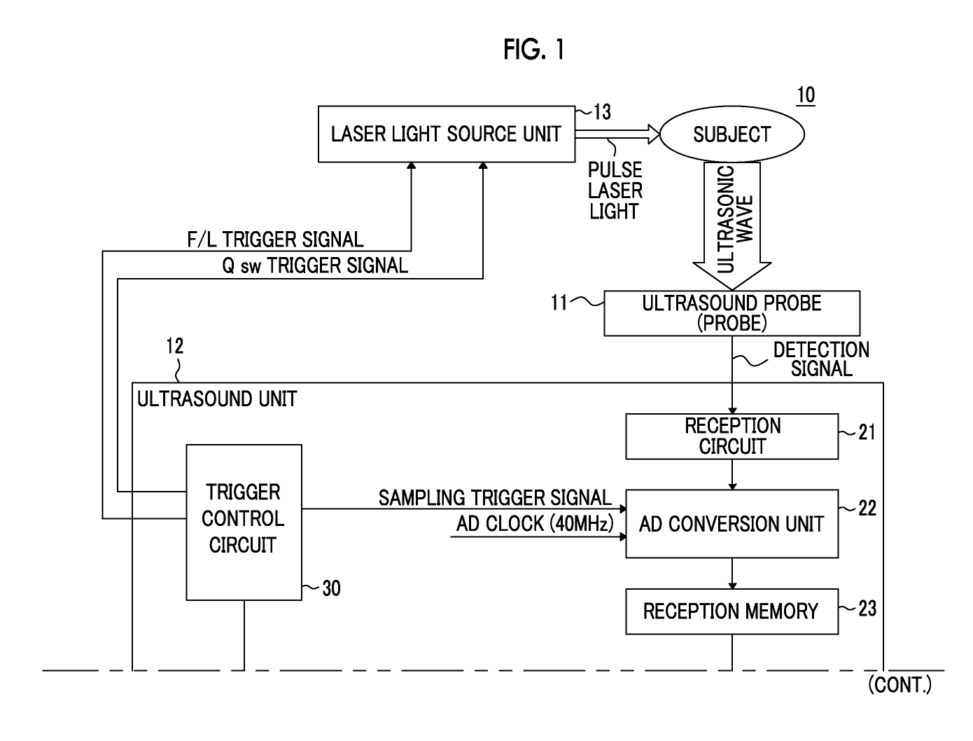

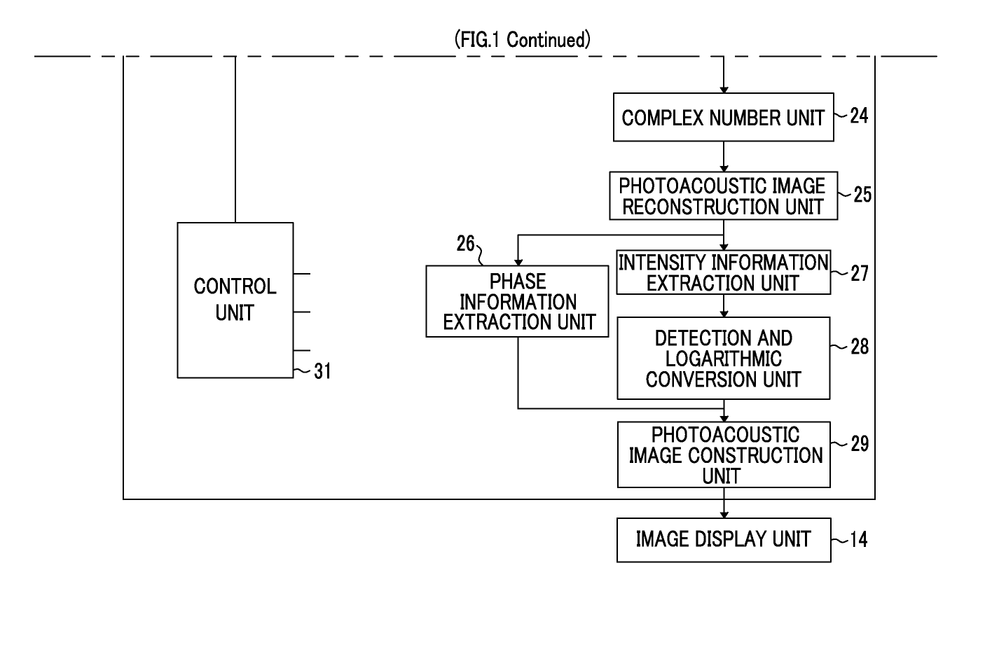

Hereinafter, an embodiment of the invention will be described in detail referring to the drawings. FIG. 1 shows a photoacoustic measurement device including a laser device according to a first embodiment of the invention. A photoacoustic measurement device 10 includes an ultrasound probe (probe) 11, an ultrasound unit 12, and a laser light source unit (laser device) 13. In the embodiment of the invention, although an ultrasonic wave is used as an acoustic wave, the acoustic wave is not limited to the ultrasonic wave, and an acoustic wave having an audible frequency may be used insofar as an appropriate frequency is selected depending on an object to be examined or a measurement condition.

The laser light source unit 13 emits pulse laser light to be irradiated onto a subject. The laser light source unit 13 emits laser light having a plurality of wavelengths including first and second wavelengths. The second wavelength is shorter than the first wavelength. In a laser gain coefficient wavelength characteristic, a gain coefficient at the second wavelength is higher than a gain coefficient at the first wavelength. For example, the laser gain coefficient has a maximum value at the second wavelength, monotonously decreases with a decrease in wavelength in a range of wavelength shorter than the second wavelength, and monotonously decreases with an increase in wavelength in a range of wavelength longer than the second wavelength.

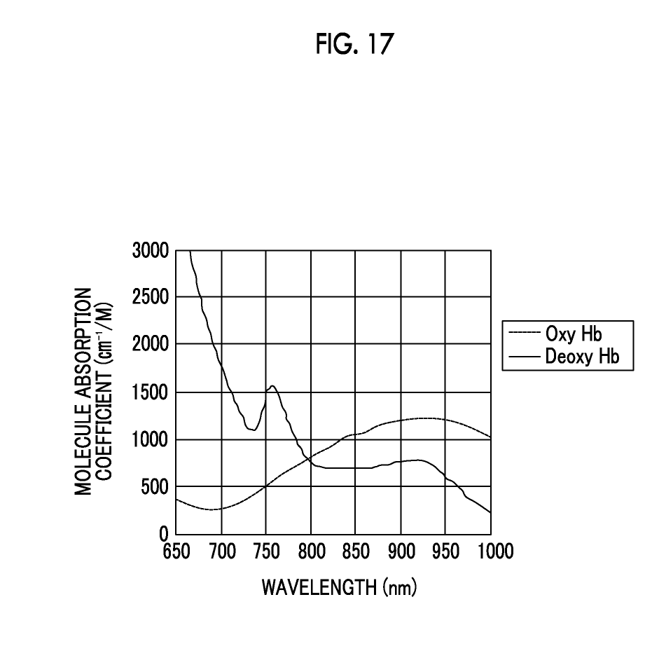

For example, the first wavelength (center wavelength) of about 800 nm is considered, and the second wavelength of about 750 nm is considered. Referring to FIG. 17 described above, a molecular absorption coefficient at a wavelength of 750 nm of oxygenated hemoglobin (hemoglobin bonded to oxygen: oxy-Hb) contained in a large amount in a human artery is lower than a molecular absorption coefficient at a wavelength of 800 nm. A molecular absorption coefficient at a wavelength of 750 nm of deoxygenated hemoglobin (hemoglobin not bonded to oxygen: deoxy-Hb) contained in a large amount in a vein is higher than a molecular absorption coefficient at a wavelength of 800 nm. This nature is use to check whether a photoacoustic signal obtained at the wavelength of 750 nm is relatively greater or smaller than a photoacoustic signal obtained at the wavelength of 800 nm, thereby distinguishing between the photoacoustic signal from the artery and the photoacoustic signal from the vein.

In regard to the selection of the first wavelength and the second wavelength, in theory, any combination of two wavelengths may be used insofar as there is a difference in optical absorption coefficient between two wavelengths to be selected, and the invention is not limited to the combination of about 750 nm and about 800 nm. Considering ease of handling or the like, it is preferable that the two wavelengths to be selected are a combination of a wavelength of about 800 nm (accurately, 798 nm) at which the optical absorption coefficient is the same between oxygenated hemoglobin and deoxygenated hemoglobin and a wavelength of about 750 nm (accurately, 757 nm) at which the optical absorption coefficient of deoxygenated hemoglobin has a maximum value. The first wavelength does not need to be accurately 798 nm, and for example, if the first wavelength is in a range of 793 nm to 802 nm, there is no practical problem. The second wavelength does not need to be accurately 757 nm, and for example, if the second wavelength is in a range of 748 nm to 770 nm which is a half width of a peak around the maximum value (757 nm), there is no practical problem.

For example, laser light emitted from the laser light source unit 13 is guided to the probe 11 by means of a light guide, such as an optical fiber, and is irradiated from the probe 11 toward the subject. The irradiation position of laser light is not particularly limited, and laser light may be irradiated from a place other than the probe 11. In the subject, an optical absorber absorbs energy of irradiated laser light, whereby an ultrasonic wave (acoustic wave) is generated. The probe 11 includes an ultrasonic detector. For example, the probe 11 has a plurality of ultrasonic detector elements (ultrasound transducers) arranged in a one-dimensional manner, and detects an acoustic wave (photoacoustic signal) from the subject by the ultrasound transducers arranged in a one-dimensional manner.

The ultrasound unit 12 has a reception circuit 21, an AD conversion unit 22, a reception memory 23, a complex number unit 24, a photoacoustic image reconstruction unit 25, a phase information extraction unit 26, an intensity information extraction unit 27, a detection and logarithmic conversion unit 28, a photoacoustic image construction unit 29, a trigger control circuit 30, and a control unit 31. The reception circuit 21 receives the photoacoustic signal detected by the probe 11. The AD conversion unit 22 is a detection unit, samples the photoacoustic signal received by the reception circuit 21, and generates photoacoustic data which is digital data. The AD conversion unit 22 performs sampling of the photoacoustic signal in a predetermined sampling period in synchronization with an AD clock signal.

The AD conversion unit 22 stores photoacoustic data in the reception memory 23. The AD conversion unit 22 stores photoacoustic data corresponding to each wavelength of pulse laser light emitted from the laser light source unit 13 in the reception memory 23. That is, the AD conversion unit 22 stores, in the reception memory 23, first photoacoustic data obtained by sampling the photoacoustic signal detected by the probe 11 when pulse laser light having the first wavelength is irradiated onto the subject and second photoacoustic data obtained by sampling the photoacoustic signal detected by the probe 11 when pulse laser light having the second wavelength is irradiated.

The complex number unit 24 reads the first photoacoustic data and the second photoacoustic data from the reception memory 23, and generates complex data in which one of the first photoacoustic data and the second photoacoustic data is a real part and the other data is an imaginary part. Hereinafter, a case where the complex number unit 24 generates complex data in which the first photoacoustic data is an imaginary part and the second photoacoustic data is a real part will be described.

The photoacoustic image reconstruction unit 25 receives complex data from the complex number unit 24 as input. The photoacoustic image reconstruction unit 25 performs image reconstruction from input complex data by a Fourier transformation method (FTA method). In the image reconstruction by the Fourier transformation method, for example, a known method in the related art described in “Photoacoustic Image Reconstruction-A Quantitative Analysis” Jonathan I. Sperl et al. SPIE-OSA Vol. 6631 663103 or the like can be applied. The photoacoustic image reconstruction unit 25 inputs Fourier transformed data representing a reconstructed image to the phase information extraction unit 26 and the intensity information extraction unit 27.

The phase information extraction unit 26 extracts the magnitude relationship of relative signal intensity between photoacoustic data corresponding to the respective wavelengths. In this embodiment, the phase information extraction unit 26 has a reconstructed image reconstructed by the photoacoustic image reconstruction unit 25 as input data and generates phase information, which represents how much one of the real part and the imaginary part is relatively greater than the other part when comparing, from input data as complex data. For example, when complex data is expressed by X+iY, the phase information extraction unit 26 generates θ=tan⁻¹(Y/X) as the phase information. When X=0, θ=90°. When the second photoacoustic data (X) constituting the real part and the first photoacoustic data (Y) constituting the imaginary part are equal to each other, the phase information becomes θ=45°. The phase information becomes close to θ=0° as the second photoacoustic data is relatively greater, and becomes close to θ=90° as the first photoacoustic data is greater.

The intensity information extraction unit 27 generates intensity information representing signal intensity based on the photoacoustic data corresponding to the respective wavelengths. In this embodiment, the intensity information extraction unit 27 has the reconstructed image reconstructed by the photoacoustic image reconstruction unit 25 as input data and generates the intensity information from input data as complex data. For example, when complex data is expressed by X+iY, the intensity information extraction unit 27 extracts (X²+Y²)^{1/2} as the intensity information. The detection and logarithmic conversion unit 28 generates an envelope of data representing the intensity information extracted by the intensity information extraction unit 27 and logarithmically converts the envelope to expand a dynamic range.

The photoacoustic image construction unit 29 receives the phase information from the phase information extraction unit 26 as input and receives intensity information after detection and logarithmic conversion processing from the detection and logarithmic conversion unit 28 as input. The photoacoustic image construction unit 29 generates a photoacoustic image, which is a distribution image of an optical absorber, based on the input phase information and intensity information. The photoacoustic image construction unit 29 determines the luminance (gradation value) of each pixel in the distribution image of the optical absorber based on the input intensity information. The photoacoustic image construction unit 29 determines the color (display color) of each pixel in the distribution image of the optical absorber based on the phase information. The photoacoustic image construction unit 29 determines the color of each pixel based on the input phase information by means of a color map, in which a phase range of 0° to 90° corresponds to predetermined colors.

Since a phase range of 0° to 45° is a range in which the second photoacoustic data is greater than the first photoacoustic data, it is considered that the generation source of the photoacoustic signal is a vein in which blood primarily containing deoxygenated hemoglobin having greater absorption for the wavelength of 756 nm than absorption for the wavelength of 798 nm flows. Since a phase range of 45° to 90° is a range in which the second photoacoustic data is smaller than the first photoacoustic data, it is considered that the generation source of the photoacoustic signal is an artery in which blood primarily containing oxygenated hemoglobin having smaller absorption for the wavelength of 756 nm than absorption for the wavelength of 798 nm flows.

Accordingly, as the color map, for example, a color map in which the phase 0° is blue, color gradually changes to be colorless (white) as the phase becomes close to 45°, the phase 90° is red, and color gradually changes to be white as the phase becomes close to 45° is used. In this case, on the photoacoustic image, a portion corresponding to an artery can be expressed in red, and a portion corresponding to a vein can be expressed in blue. The gradation value may be constant, and color coding of the portion corresponding to the artery and the portion corresponding to the vein may be merely performed according to the phase information without using the intensity information. An image display unit 14 displays the photoacoustic image generated by the photoacoustic image construction unit 29 on a display screen.

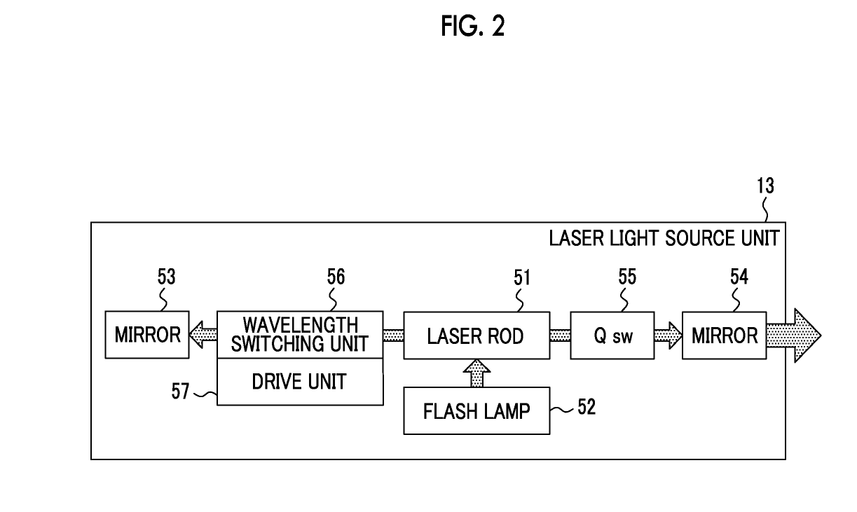

Next, the configuration of the laser light source unit 13 will be described in detail. FIG. 2 shows the configuration of the laser light source unit 13. The laser light source unit 13 has a laser rod 51, a flash lamp 52, mirrors 53 and 54, a Q switch 55, a wavelength switching unit 56, and a drive unit 57. The laser rod 51 is a laser medium. For the laser rod 51, for example, alexandrite crystal may be used. The laser gain coefficient at the first wavelength (800 nm) of the alexandrite crystal is lower than the laser gain coefficient at the second wavelength (750 nm). The flash lamp 52 is an excitation light source, and irradiates excitation light onto the laser rod 51. A light source other than the flash lamp 52 may be used as an excitation light source.

The mirrors 53 and 54 face each other with the laser rod 51 interposed therebetween, and an optical resonator is configured by the mirrors 53 and 54. It is assumed that the mirror 54 is on an output side. In the optical resonator, the Q switch 55 and the wavelength switching unit 56 are inserted. Insertion loss in the optical resonator is rapidly changed from great loss (low Q) to small loss (high Q) by the Q switch 55, thereby obtaining pulse laser light. The wavelength switching unit 56 is used when switching the wavelength of light oscillating in the optical resonator between the first wavelength and the second wavelength. The wavelength switching unit 56 includes a first long path filter which transmits light having a wavelength equal to or greater than the first wavelength.

The drive unit 57 drives the wavelength switching unit 56. The drive unit 57 drives the wavelength switching unit 56 to insert the first long path filter on the optical path of the optical resonator when the wavelength of laser light to be emitted from the laser light source unit 13 is the first wavelength. In this case, the wavelength switching unit 56 inhibits the transmission of a component having a wavelength shorter than the first wavelength among light emitted from the laser rod 51. The drive unit 57 drives the wavelength switching unit 56 to remove the first long path filter from the optical path of the optical resonator when the wavelength of laser light to be emitted is the second wavelength. In this case, the wavelength switching unit 56 transmits light having the first and second wavelengths, for example, all wavelength components of light emitted from the laser rod 51.

Returning to FIG. 1, the control unit 31 performs control of the respective units in the ultrasound unit 12. The trigger control circuit 30 outputs a flash lamp trigger signal for controlling the emission of the flash lamp 52 (FIG. 2) to the laser light source unit 13 and causes excitation light to be irradiated from the flash lamp 52 onto the laser rod 51. The trigger control circuit 30 outputs a Q switch trigger signal to the Q switch 55 after outputting the flash lamp trigger signal. The Q switch 55 rapidly changes insertion loss in the optical resonator from great loss to small loss in response to the Q switch trigger signal (the Q switch is turned on), whereby pulse laser light is emitted from the output mirror 54.

The trigger control circuit 30 outputs a sampling trigger signal (AD trigger signal) to the AD conversion unit 22 in conformity with the timing of the Q switch trigger signal, that is, the emission timing of pulse laser light. The AD conversion unit 22 starts sampling of the photoacoustic signal based on the sampling trigger signal.

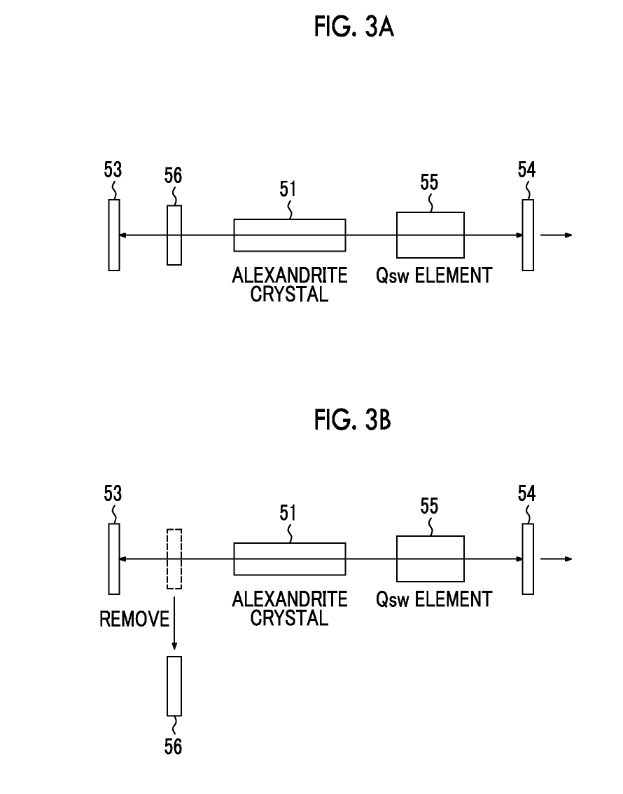

Subsequently, wavelength switching in the laser light source unit 13 will be described. FIGS. 3A and 3B show the internal configuration of the optical resonator of the laser light source unit 13. The wavelength switching unit 56 is configured as a long path filter which transmits light having a wavelength equal to or greater than 800 nm. For example, if a wavelength at which transmittance of the long path filter becomes 50% is defined as a cutoff wavelength, for the wavelength switching unit 56, a long path filter in which a wavelength slightly shorter than the wavelength of 800 nm is a cutoff wavelength is used. For example, a long path filter has a wavelength characteristic of light transmittance, such as a wavelength characteristic in which light transmittance may not become high enough to be considered total transmission (light transmittance is substantially 100%) in a wavelength range shorter than 800 nm around the wavelength of 800 nm and light may be substantially total-transmitted initially if the wavelength becomes 800 nm.

FIG. 3A shows a state where the wavelength switching unit (long path filter) 56 is inserted on the optical path of the optical resonator. The drive unit 57 displaces the position of the long path filter 56 by, for example, a motor or the like when the wavelength of laser light to be emitted is the first wavelength (800 nm) and inserts the long path filter 56 on the optical path of the optical resonator. FIG. 3B shows a state where the long path filter 56 is removed from the optical path of the optical resonator. The drive unit 57 moves the long path filter 56 outside the optical path of the optical resonator by a motor or the like when the wavelength of laser light to be emitted is the second wavelength (750 nm).

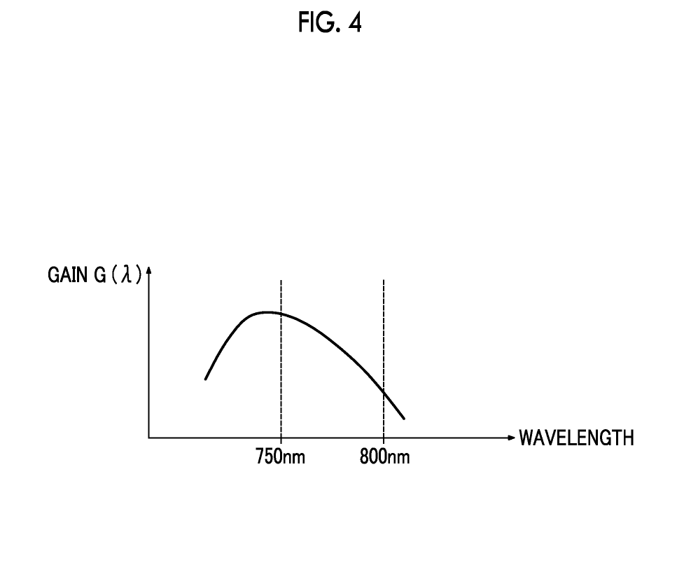

FIG. 4 shows a gain of alexandrite. A gain coefficient g(λ,T) of alexandrite is expressed by the following expression.

$$g ( λ , T ) = {N}_{0} ( p - ( 1 - p ) exp ( \frac{E - {E}_{zpl}}{kT} ) ) {σ}_{em} ( λ , T ) \qquad ( 1 )$$

Here, p is a function of an inverted distribution rate (the number of upper levels/addition concentration). p is in proportion to excitation energy. Ezpl is zero-phonon energy. The gain G(λ) of alexandrite is expressed by the following expression when l_{rod} is the length of an alexandrite rod.

G(λ)=exp[g(λ,T)×l_{rod}])

As shown in FIG. 4, the laser gain G(λ) of alexandrite has a peak around a wavelength of 750 nm and decreases as the wavelength becomes longer in a wavelength range exceeding the wavelength of 750 nm.

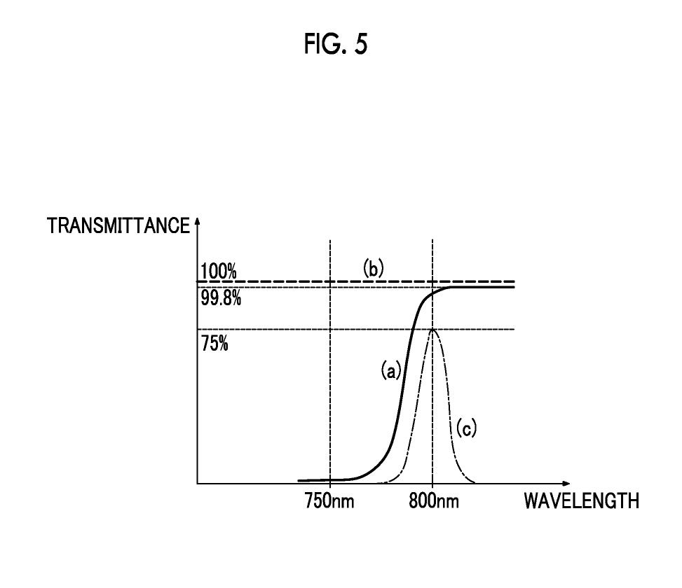

FIG. 5 shows the light transmittance of the wavelength switching unit 56. In FIG. 5, a graph (a) shows the wavelength characteristic of light transmittance of the long path filter for use in the wavelength switching unit 56, and a graph (b) shows the wavelength characteristic of light transmittance at the position of the wavelength switching unit 56 in a state where the wavelength switching unit 56 is removed from the optical path of the optical resonator (FIG. 3B). As shown in the graph (a), the wavelength switching unit (long path filter) 56 transmits light having a wavelength of 800 nm at high light transmittance of, for example, 99.8%, and hardly transmits light having a wavelength of 750 nm. When the long path filter is removed from the optical path of the optical resonator, since there is no particular member, which blocks light, on the optical path of the optical resonator, light having the wavelength of 750 nm and light having the wavelength of 800 nm are substantially transmitted directly (100%).

In FIG. 5, as a comparative example, the wavelength characteristic of light transmittance of a bandpass filter, which selectively transmits light having a wavelength of 800 nm is shown in a graph (c). Even when the bandpass filter having the wavelength characteristic of light transmittance shown in the graph (c) is used, as when the long path filter is used, light having a wavelength of 800 nm can be transmitted and light having a wavelength of 750 nm can be blocked. However, the light transmittance of the bandpass filter is about 75% at most, and light transmission is deteriorated compared to the light transmittance of the long path filter, and the amount of light to be transmitted decreases compared to when the long path filter is used.

Total loss in the optical resonator can be expressed by the following expression when the above-described light transmittance is T(λ), R₁ and R₂ are respectively reflectance of the mirrors 53 and 54, and L is internal loss of the optical resonator.

Loss(λ)=|−ln R₁R₂T(λ)²+L|/2

The effective gain g_{eff} of the optical resonator is obtained by subtracting the total loss in the optical resonator from the gain of alexandrite.

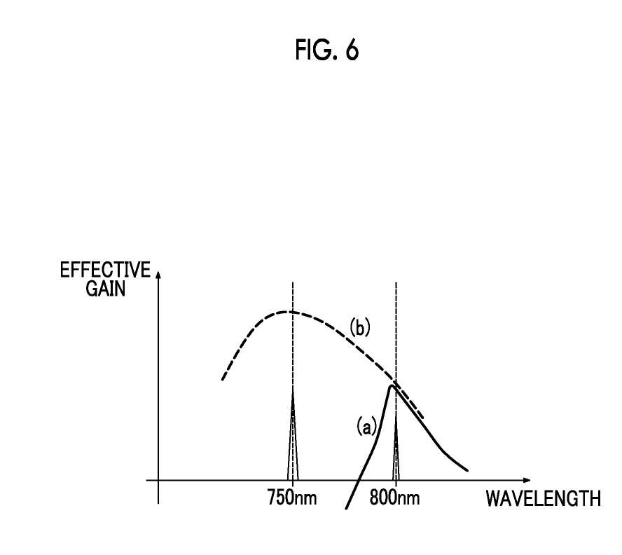

FIG. 6 shows the effective gain of the optical resonator. In FIG. 6, a graph (a) represents an effective gain when the long path filter having the wavelength characteristic shown in the graph (a) of FIG. 5 is inserted on the optical path of the optical resonator, and a graph (b) represents an effective gain when the long path filter is removed. When the long path filter is not inserted (FIG. 3B), as shown in the graph (b) of FIG. 6, the effective gain is maximal around a wavelength of 750 nm as in the wavelength characteristic (FIG. 4) of the laser gain of alexandrite. Laser oscillation occurs at a point (wavelength, excitation power) at which the effective gain >0. When increasing the excitation power, the effective gain is initially greater than 0 at the wavelength of 750 nm at which the effective gain is highest. Accordingly, when the long path filter is not inserted on the optical path of the optical resonator, the optical resonator oscillates at the wavelength of 750 nm of the peak position in the wavelength characteristic of the effective gain.

When the long path filter is inserted on the optical path of the optical resonator (FIG. 3A), on a wavelength side shorter than the cutoff wavelength of the long path filter, since loss in the optical resonator is great, the effective gain is low, and the effective gain is maximal around the wavelength of 800 nm at which the long path filter transmits light at high light transmittance. Accordingly, when the long path filter is inserted, the optical resonator oscillates at the wavelength of 800 nm of the peak position in the wavelength characteristic of the effective gain.

In the above description, although the oscillation wavelength is switched between 800 nm and 750 nm according to whether the long path filter is inserted on the optical path of the optical resonator or the long path filter is removed from the optical path, the invention is not limited thereto. For example, the wavelength switching unit 56 may have an optical member, which transmits light having at least a wavelength of 750 nm, in addition to the long path filter, and the optical member may be inserted on the optical path of the optical resonator when the wavelength of laser light is 750 nm.

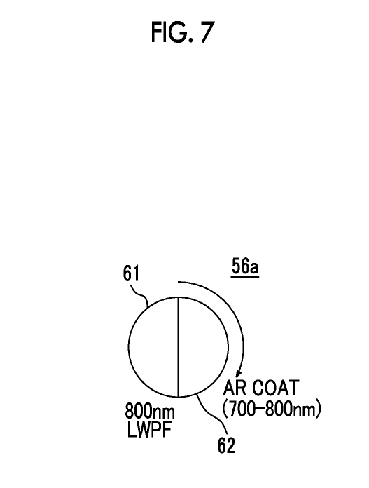

FIG. 7 shows a modification example of the wavelength switching unit 56. In this example, the wavelength switching unit 56 is configured as a filter rotor which inserts the long path filter on the optical path of the optical resonator and removes the long path filter from the optical path of the optical resonator with rotation displacement. A wavelength switching unit (filter rotor) 56a has a first region 61 where the long path filter is disposed and a second region 62 where an optical member which substantially transmits light in the total wavelength bands directly is disposed. For example, a region from the rotation displacement position of 0° to 180° corresponds to the first region 61 where the long path filter is disposed, and a region from the rotation displacement position of 180° to 360° corresponds to the second region 62 where the optical member is disposed.

The filter rotor 56a is attached to the output shaft of a servo motor as the drive unit 57 (FIG. 2), and is driven to rotate with the rotation of the servo motor. The rotation displacement of the filter rotor 56a can be detected by means of a rotary encoder including a slitted rotary plate attached to the output shaft of the servo motor and a transmissive photointerrupter. For example, a voltage supplied to the servo motor, or the like is controlled such that the amount of rotation displacement of the rotary shaft of the servo motor detected by the rotary encoder for a predetermined time is maintained to a predetermined amount, whereby the filter rotor 56a can be rotated at a given speed. The filter rotor 56a is driven to rotate consecutively, whereby the long path filter and the optical member can be alternately inserted on the optical resonator.

For the above-described optical member, an optical member having high light transmittance, such as glass, may be used. It is preferable that, on the optical member, an anti-reflection film which does not reflect light having at least a wavelength of 750 nm, for example, an anti-reflection film which does not reflect light in a wavelength range of 700 nm to 800 nm is formed. When the first region 61 is located on the optical path of the optical resonator, the long path filter disposed in the first region 61 cuts light in a wavelength band shorter than a wavelength of 800 nm, whereby the effective gain of the optical resonator in a wavelength band shorter than the wavelength of 800 nm decreases, and laser light having the wavelength of 800 nm can be obtained. When the second region 62 is located on the optical path of the optical resonator, since the second region 62 does not particularly cut light in a specific wavelength band, laser light having the wavelength of 750 nm, at which the gain coefficient of alexandrite is maximal, can be obtained.

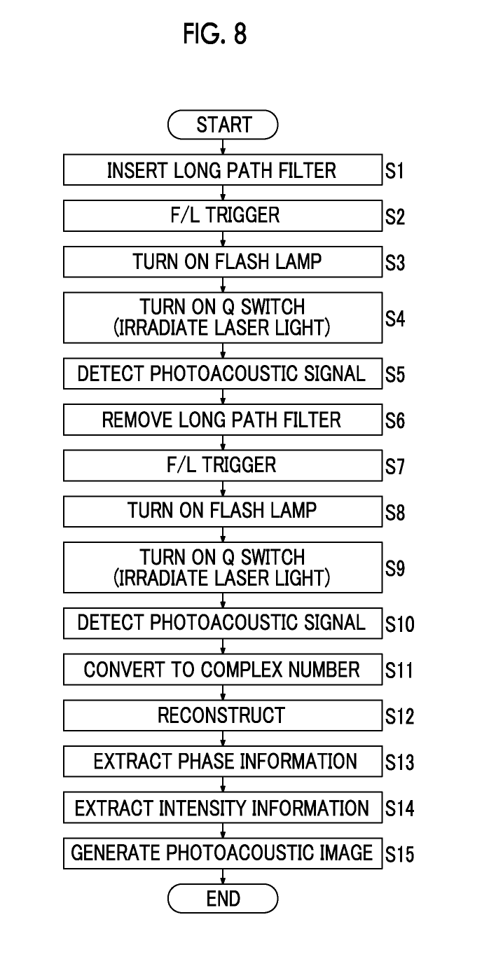

Subsequently, an operation procedure will be described. FIG. 8 shows an operation procedure of the photoacoustic measurement device 10. The drive unit 57 (FIG. 2) drives the wavelength switching unit 56 to insert the long path filter, which transmits light having a wavelength equal to or greater than 800 nm, on the optical path of the optical resonator (Step S1). For example, as shown in FIG. 3A, the drive unit 57 inserts the wavelength switching unit 56 configured as the long path filter on the optical path of the optical resonator. Alternatively, as shown in FIG. 6, the wavelength switching unit 56 is configured as the filter rotor 56a having the first region 61 where the long path filter is disposed and the second region 62 where the optical member is disposed, the drive unit 57 rotates and drives the filter rotor 56a such that the first region 61 is inserted on the optical path of the optical resonator.

If the reception of the photoacoustic signal is prepared, the trigger control circuit 30 (FIG. 1) outputs the flash lamp trigger signal to the laser light source unit 13 to emit the pulse laser light having the first wavelength (800 nm) (Step S2). The flash lamp 52 of the laser light source unit 13 is turned on in response to the flash lamp trigger signal, and the excitation of the laser rod 51 starts (Step S3).

The trigger control circuit 30 outputs the Q switch trigger signal at a predetermined timing after the flash lamp 52 is turned on, and turns on the Q switch 55 (Step S4). The Q switch 55 is turned on, whereby the laser light source unit 13 emits pulse laser light having the wavelength of 800 nm. When the wavelength switching unit 56 has the filter rotor shown in FIG. 6 and the filter rotor is driven to rotate consecutively, the trigger control circuit 30 may turn on the Q switch at the timing at which the filter rotor inserts the first region 61 on the optical path of the optical resonator.

Pulse laser light having the wavelength of 800 nm emitted from the laser light source unit 13 is guided to, for example, the probe 11 and irradiated from the probe 11 onto the subject. In the subject, the optical absorber absorbs energy of irradiated pulse laser light, whereby a photoacoustic signal is generated. The probe 11 detects the photoacoustic signal generated in the subject. The photoacoustic signal detected by the probe 11 is received by the reception circuit 21.

The trigger control circuit 30 outputs the sampling trigger signal to the AD conversion unit 22 in conformity with the output timing of the Q switch trigger signal. The AD conversion unit 22 samples the photoacoustic signal received by the reception circuit 21 in a predetermined sampling period (Step S5). The photoacoustic signal sampled by the AD conversion unit 22 is stored as first photoacoustic data in the reception memory 23.

After pulse laser light having the wavelength of 800 nm is emitted, the drive unit 57 drives the wavelength switching unit 56 to remove the long path filter from the optical path of the optical resonator (Step S6). For example, as shown in FIG. 3B, the drive unit 57 moves the wavelength switching unit 56 configured as the long path filter outside the optical path of the optical resonator. Alternatively, as shown in FIG. 6, when the wavelength switching unit 56 is configured as the filter rotor 56a having the first region 61 where the long path filter is disposed and the second region 62 where the optical member is disposed, the drive unit 57 rotates and drives the filter rotor 56a such that the second region 62 is inserted on the optical path of the optical resonator.

If the reception of the photoacoustic signal is prepared, the trigger control circuit 30 outputs the flash lamp trigger signal to the laser light source unit 13 to emit pulse laser light having the second wavelength (750 nm) (Step S7). The flash lamp 52 of the laser light source unit 13 is turned on in response to the flash lamp trigger signal, and the excitation of the laser rod 51 starts (Step S8).

The trigger control circuit 30 outputs the Q switch trigger signal at a predetermined timing after the flash lamp 52 is turned on, and turns on the Q switch 55 (Step S9). The Q switch 55 is turned on, whereby the laser light source unit 13 emits pulse laser light having the wavelength of 750 nm. When wavelength switching unit 56 is configured as the filter rotor shown in FIG. 6 and the filter rotor is driven to rotate consecutively, the trigger control circuit 30 may turn on the Q switch at the timing at which the filter rotor inserts the second region 62 on the optical path of the optical resonator.

Pulse laser light having the wavelength of 750 nm emitted from the laser light source unit 13 is guided to, for example, the probe 11 and irradiated from the probe 11 onto the subject. In the subject, the optical absorber absorbs energy of irradiated pulse laser light, whereby a photoacoustic signal is generated. The probe 11 detects the photoacoustic signal generated in the subject. The photoacoustic signal detected by the probe 11 is received by the reception circuit 21.

The trigger control circuit 30 outputs the sampling trigger signal to the AD conversion unit 22 in conformity with the output timing of the Q switch trigger signal. The AD conversion unit 22 samples the photoacoustic signal received by the reception circuit 21 in a predetermined sampling period (Step S10). The photoacoustic signal sampled by the AD conversion unit 22 is stored as second photoacoustic data in the reception memory 23.

The first and second photoacoustic data are stored in the reception memory, whereby data necessary for generating a photoacoustic image for one frame is gathered. When a range in which a photoacoustic image is generated is divided into a plurality of partial regions, the processing of Steps S1 to S10 may be executed for each partial region.

The complex number unit 24 reads the first photoacoustic data and the second photoacoustic data from the reception memory 23, and generates complex data in which the first photoacoustic data is an imaginary part and the second photoacoustic data is a real part (Step S11). The photoacoustic image reconstruction unit 25 performs image reconstruction from complex data converted to a complex number in Step S11 by a Fourier transformation method (FTA method) (Step S12).

The phase information extraction unit 26 extracts phase information from the reconstructed complex data (reconstructed image) (Step S13). For example, when the reconstructed complex data is expressed by X+iY, the phase information extraction unit 26 extracts θ=tan⁻¹(Y/X) as the phase information (where, when X=0, θ=90°). The intensity information extraction unit 27 extracts intensity information from the reconstructed complex data (Step S14). For example, when the reconstructed complex data is expressed by X+iY, the intensity information extraction unit 27 extracts (X²+Y²)^{1/2}) as the intensity information.

The detection and logarithmic conversion unit 28 carries out the detection and logarithmic conversion processing for the intensity information extracted in Step S14. The photoacoustic image construction unit 29 generates a photoacoustic image based on the phase information extracted in Step S13 and the result of the detection and logarithmic conversion processing for the intensity information extracted in Step S14 (Step S15). For example, the photoacoustic image construction unit 29 determines the luminance (gradation value) of each pixel in the distribution image of the optical absorber based on the intensity information and determines the color of each pixel based on the phase information, thereby generating the photoacoustic image. The generated photoacoustic image is displayed on the image display unit 14.

The laser light source unit 13 of this embodiment can emit light having the first wavelength and light having the second wavelength shorter than the first wavelength. The laser gain coefficient at the second wavelength is higher than the gain coefficient at the first wavelength. Conversely, the laser gain coefficient at the first wavelength is lower than the laser gain coefficient at the second wavelength. The wavelength switching unit 56 includes the long path filter which transmits light having a wavelength equal to or greater than the first wavelength, and inserts the long path filter on the optical path of the optical resonator when the wavelength of laser light to be emitted is the first wavelength. The long path filter is inserted on the optical path of the optical resonator, the effective gain at the second wavelength of the optical resonator decreases. If the laser gain decreases as the wavelength become longer from the second wavelength to the first wavelength, the effective gain of the optical resonator is maximal at the first wavelength in a state where the long path filter is inserted, and the optical resonator oscillates at the first wavelength, thereby obtaining laser light having the first wavelength.

When the wavelength of laser light to be emitted is the second wavelength, the wavelength switching unit 56 does not insert the long path filter on the optical path of the optical resonator. In this case, if the laser gain has a maximum value at the second wavelength in the wavelength characteristic of the laser gain, the optical resonator can oscillate at the second wavelength, and laser light having the second wavelength can be obtained. In this way, the wavelength of laser light can be switched according to whether or not to insert the long path filter on the optical path of the optical resonator. In general, the long path filter has high light transmittance compared to the bandpass filter, does not decrease laser efficiency, and can allow wavelength switching. The long path filter can be manufactured at lower cost and has a simple configuration. For this reason, cost can be reduced compared to a birefringent filter made of quartz is used.

In this embodiment, complex data in which one of the first photoacoustic data and the second photoacoustic data obtained at the two wavelengths is a real part and the other data is an imaginary part is generated, and the reconstructed image is generated from complex data by the Fourier transformation method. In this case, it is possible to efficiently perform reconstruction compared to a case where the first photoacoustic data and the second photoacoustic data are separately reconstructed. Pulse laser light having a plurality of wavelengths is irradiated, and the photoacoustic signals (photoacoustic data) when pulse laser light having the respective wavelengths is irradiated are used, whereby it is possible to perform functional imaging using the fact that the optical absorption characteristics of the optical absorbers differ depending on wavelength.

Subsequently, a second embodiment of the invention will be described. In the first embodiment, the long path filter is not inserted on the optical path of the optical resonator when the wavelength of laser light to be emitted is the second wavelength, and the optical resonator naturally oscillates around the wavelength of 750 nm (free running). The alexandrite crystal undergoes change in laser gain with change in temperature or the like, and the oscillation center wavelength of 750 nm changes by about several nm. For example, in an application, such as photoacoustics, in which wavelength precision is important, it is not preferable since signal quality is deteriorated due to fluctuation in wavelength by several nm.

As described above, laser light having the second wavelength undergoes fluctuation in oscillation wavelength with change in temperature or the like. In regard to the first wavelength, the oscillation wavelength is determined by the wavelength characteristic of light transmittance of the long path filter which is inserted on the optical path of the optical resonator by the wavelength switching unit 56, and even when the laser gain changes with change in temperature, the oscillation wavelength of the optical resonator does not fluctuate. In this embodiment, a separate long path filter which transmits light having a wavelength equal to or greater than the second wavelength is used, and the separate long path filter is inserted on the optical path of the optical resonator when the wavelength of laser light to be emitted is the second wavelength, whereby the oscillation wavelength is also stabilized for the second wavelength.

In this embodiment, the wavelength switching unit 56 (FIG. 2) has a second long path filter which transmits light having a wavelength equal to or greater than the second wavelength, in addition to the long path filter (first long path filter) which transmits light having a wavelength equal to or greater than the first wavelength. The wavelength switching unit 56 inserts the first long path filter on the optical path of the optical resonator when the wavelength of laser light to be emitted is the first wavelength. When the wavelength of laser light to be emitted is the second wavelength, the second long path filter is inserted on the optical path of the optical resonator.

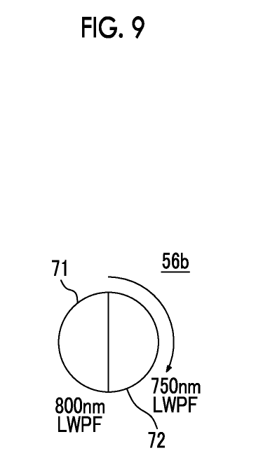

FIG. 9 shows a configuration example of a wavelength switching unit. In this example, a wavelength switching unit is configured as a filter rotor which inserts the first or second long path filter on the optical path of the optical resonator with rotation displacement. A wavelength switching unit (filter rotor) 56b has a first region 71 where the first long path filter is disposed and a second region 72 where the second long path filter is disposed. For example, a region from the rotation displacement position of 0° to 180° corresponds to the first region 71 where the first long path filter is disposed, and a region from the rotation displacement position of 180° to 360° corresponds to the second region 72 where the second long path filter is disposed. For example, the drive unit 57 rotates the filter rotor 56b consecutively to alternately insert the first long path filter and the second long path filter on the optical path of the optical resonator.

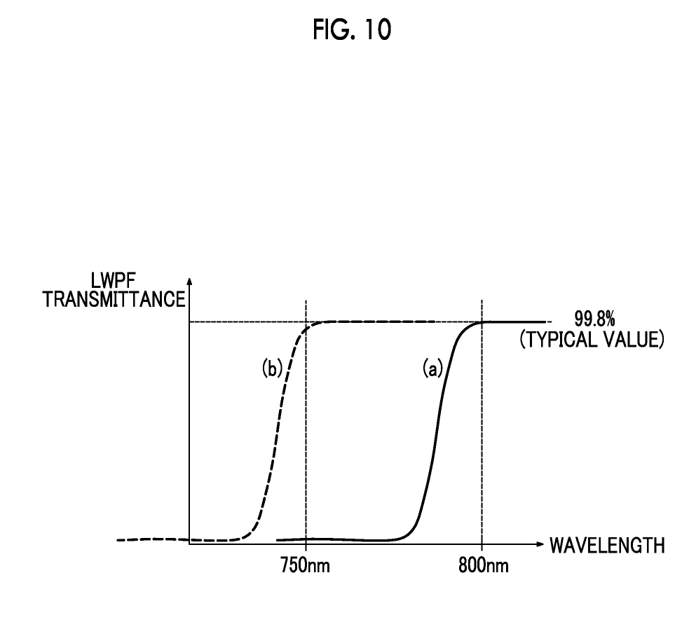

FIG. 10 shows the wavelength characteristics of light transmittance of the first and second long path filters. In FIG. 10, a graph (a) shows the wavelength characteristic of light transmittance of the first long path filter, and a graph (b) shows the wavelength characteristic of light transmittance of the second long path filter. The wavelength characteristic of light transmittance of the first long path filter is the same as that described in the first embodiment (the graph (a) of FIG. 5). As shown in the graph (b), the second long path filter transmits light having the wavelength of 750 nm at high light transmittance of, for example, 99.8% and hardly transmits light in a wavelength band shorter than the wavelength of 750 nm. The second long path filter has the wavelength characteristic of light transmittance, such as a wavelength characteristic in which light transmittance may not become high enough to be considered total transmission (light transmittance is substantially 100%) in a wavelength range shorter than the wavelength of 750 nm around the wavelength of 750 nm and light is substantially total-transmitted initially if the wavelength becomes 750 nm.

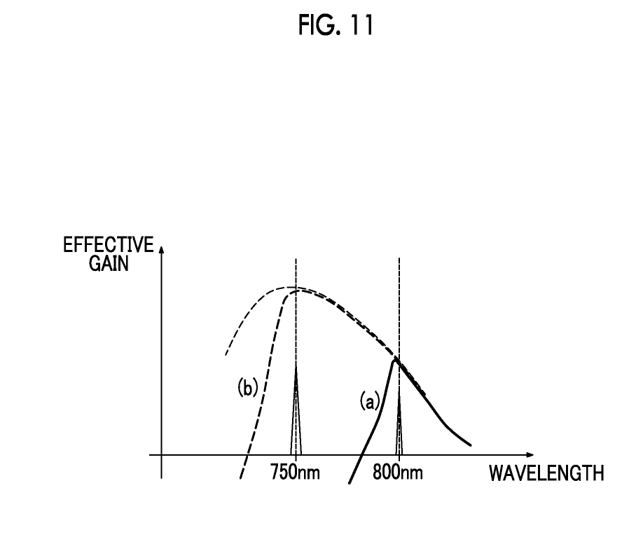

FIG. 11 shows an effective gain of an optical resonator. In FIG. 6, the graph (a) represents an effective gain when the first long path filter having the wavelength characteristic shown in the graph (a) of FIG. 10 is inserted on the optical path of the optical resonator, and the graph (b) represents an effective gain when the second long path filter having the wavelength characteristic shown in the graph (b) of FIG. 10 is inserted. The wavelength characteristic of the effective gain of the optical resonator when the first long path filter is inserted on the optical path of the optical resonator is the same as that described in the first embodiment, and as shown in the graph (a), the effective gain is maximal at the wavelength of 800 nm at which the first long path filter transmits light initially at high light transmittance.

When the second long path filter is inserted on the optical path of the optical resonator, on a wavelength side shorter than the cutoff wavelength of the second long path filter, since loss in the optical resonator is great, the effective gain decreases compared to the execution efficiency of the optical resonator indicated by a broken line in FIG. 11 when no filter is inserted. On a wavelength side longer than the wavelength of 750 nm, since the light transmittance of the second long path filter is high to be, for example, 99.8%, the effective gain is substantially the same as the execution efficiency of the optical resonator when no filter is inserted. When the second long path filter is inserted on the optical path of the optical resonator, the effective gain of the optical resonator is maximal around the wavelength of 750 nm at which the second long path filter transmits light at high light transmittance.

In this embodiment, when the wavelength of laser light to be emitted is the second wavelength, the second long path filter is inserted on the optical path of the optical resonator. The second long path filter is inserted on the optical path of the optical resonator, whereby the wavelength at which the effective gain of the optical resonator is maximal can be defined according to the wavelength characteristic of the light transmittance of the second long path filter and the oscillation wavelength at the time of laser oscillation can be controlled. In this way, the oscillation wavelength is defined by means of the second long path filter, whereby it is possible to increase wavelength stability compared to a case where natural oscillation occurs at the second wavelength. The other effects are the same as those in the first embodiment.

As shown in FIG. 4, the laser gain value has a great difference between the first wavelength (800 nm) and the second wavelength (750 nm). In this case, if the reflectance of the output mirror 54 is the same for the first wavelength and the second wavelength, the output is significantly unbalanced between the first wavelength and the second wavelength. For example, when a mirror having the reflectance of 70% which is often used at the wavelength of 750 nm of the alexandrite laser is used, while the output can be optimized at the first wavelength (750 nm), the output may significantly decrease at the second wavelength (800 nm) or oscillation may not occur. If the reflectance of the output mirror 54 is 90%, while the output can be optimized at the wavelength of 800 nm, the output is generated less at the wavelength of 750 nm.

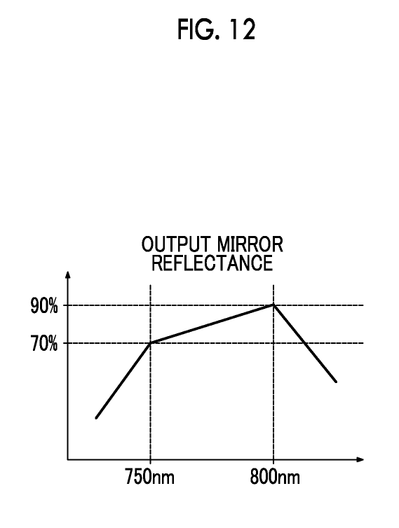

From the above-described viewpoint, it is preferable to make the reflectance of the output mirror 54 dependent on wavelength and to optimize reflectance at each wavelength. FIG. 12 shows the wavelength characteristic of reflectance of the output mirror. Since the laser gain at the first wavelength is lower than the laser gain at the second wavelength, the reflectance of the output mirror 54 for light having the first wavelength is set to be higher than the reflectance of the output mirror 54 for light having the second wavelength. Specifically, when alexandrite crystal is used for the laser rod 51, a mirror having a wavelength characteristic shown in FIG. 12, in which the reflectance for light having the wavelength of 800 nm is 90% and the reflectance for light having the wavelength of 750 nm is 70% may be used. The output mirror 54 has this wavelength characteristic, whereby the output can be optimized at both wavelengths.

As described above, if the reflectance of the output mirror 54 is set such that the optimum output is obtained at each wavelength, since the laser gain has a great difference between the first wavelength and the second wavelength, the output intensity (laser power) of laser light has a great difference between the first wavelength and the second wavelength. If laser power has a difference between the wavelengths, there is a problem in that it is necessary to correct the difference in laser power when taking the difference between the photoacoustic signals corresponding to the respective wavelengths, or the like.

In order to make the laser power at the respective wavelengths uniform, the reflectance of the output mirror 54 for light having the first wavelength and the reflectance of the output mirror 54 for light having the second wavelength may be selected such that the effective gain of the optical resonator for the first wavelength and the effective gain of the optical resonator for the second wavelength are the same. For example, the reflectance of the output mirror 54 for light having the wavelength of 800 nm is set to 90% as an optimum condition such that an optimum condition is given at 800 nm at which the laser gain is low, and the reflectance of the output mirror 54 for light having the wavelength of 750 nm is set to reflectance lower than the reflectance of 70% as an optimum condition. In this case, while the reflectance is out of the optimum condition at the wavelength of 750 nm, since the laser gain is originally high at the wavelength of 750 nm, even if the reflectance of the output mirror 54 is out of the optimum condition, there is no problem with laser oscillation. The reflectance of the output mirror 54 is set as described above, whereby it is possible to make the light intensities of laser light having both wavelengths uniform.

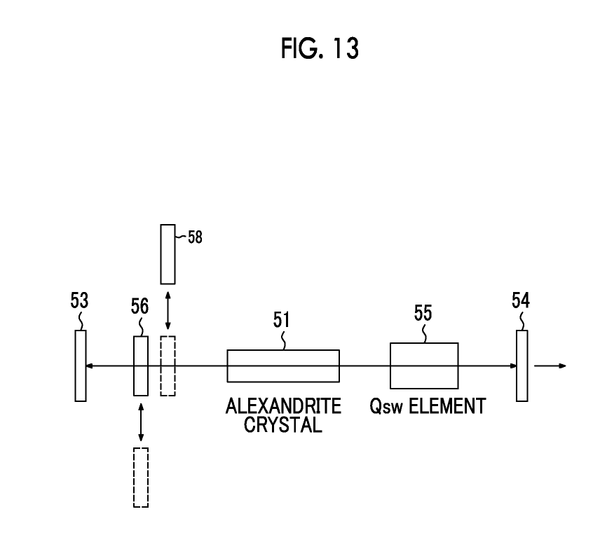

In the above description, although an example where the reflectance of the mirror changes depending on wavelength to make the light intensities of laser light uniform has been described, alternatively, when the wavelength of laser light to be emitted is the second wavelength at which the laser gain is high, a dimmer member which decreases the amount of transmission of at least light having the second wavelength may be inserted on the optical path of the optical resonator. FIG. 13 shows the internal configuration of an optical resonator of a laser light source unit according to a modification example. In this example, a dimmer filter 58 as the dimmer member is inserted on the optical path of the optical resonator. The dimmer filter 58 has transmittance from 80% to 90% for, for example, light having the wavelength of 750 nm. The dimmer filter 58 is removed from the optical path of the optical resonator when the wavelength of light to be emitted is 800 nm on a low gain side. It is preferable that the light transmittance of the dimmer filter 58 is selected such that the light intensity of light having the wavelength of 800 nm output from the laser device and the light intensity of light having the wavelength of 750 nm are the same.

As in the example shown in FIG. 7, when the wavelength switching unit 56 has an optical member which transmits at least light having the wavelength of 750 nm and the optical member is inserted on the optical path of the optical resonator when the wavelength of laser light to be emitted is 750 nm, the optical member of the wavelength switching unit 56 may double as the dimmer filter 58. The light transmittance of the dimmer filter 58 may have wavelength dependence, and the transmittance of the dimmer filter 58 for light having the wavelength of 800 nm and the transmittance of the dimmer filter 58 for light having the wavelength of 750 nm may be different. For example, the dimmer filter 58 substantially total-transmits light having the wavelength of 800 nm and attenuates and transmits a part of light having the wavelength of 750 nm. In this case, there is no need for inserting and removing the dimmer filter 58 on and from the optical path of the optical resonator.

For example, when emitting laser light having the wavelength of 800 nm, it is assumed that, if energy of 30 J is applied from the flash lamp 52 (FIG. 2) to the laser rod 51, laser light having a laser output of 100 mJ is obtained. If the oscillation wavelength is controlled to 750 nm under the same condition, when the input energy to laser rod 51 is 30 J, the effective gain of the optical resonator for the wavelength of 750 nm is higher than the gain of the optical resonator for the wavelength of 800 nm, and the laser output becomes 200 mJ. If the input energy (excitation energy) to the laser rod 51 at the wavelength of 750 nm is controlled to 15 J, the laser output becomes 100 mJ, and the laser output can be made uniform between the wavelength of 750 nm and the wavelength of 800 nm. However, in a case where the input energy increases and decreases depending on wavelength to maintain the laser output, when emitting light having the wavelength of 750 nm, if the laser rod 51 is erroneously excited by the input energy at the wavelength of 800 nm, laser light having higher light intensity than expected is emitted, and this situation is not desirable. Considering the wavelengths are switched at high speed, a mechanism which switches the output of a power supply circuit for driving the flash lamp 52 at high speed is required, and additional cost of the power supply is incurred.

As described above, when the reflectance of the mirror for light having the wavelength of 750 nm decreases or when the dimmer filter 58 is inserted on the optical path of the optical resonator at the time of the emission of light having the wavelength of 750 nm, and the laser output can be maintained constant between the wavelengths of 750 nm and 800 nm while making the input energy of the laser rod 51 constant. For example, when the input energy is set such that the light intensity of laser light at the wavelength of 800 nm on the low gain side is equal to or less than a safety specification value, even if laser oscillation is performed with the same input energy at the wavelength of 750 nm on a high gain side, it is possible to avoid the emission of laser light having light intensity, which exceeds the safety specification value of the laser. Since it is not necessary to increase and decrease the input energy depending on wavelength, the power supply circuit may drive the flash lamp 52 such that the input energy is constant, and additional cost of the power supply is not incurred.

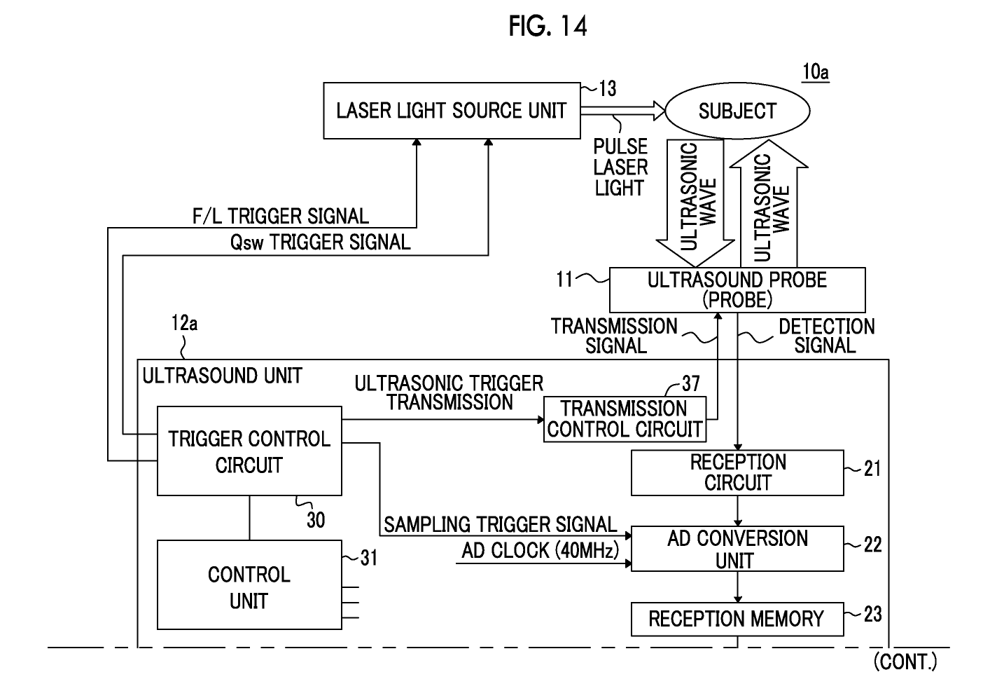

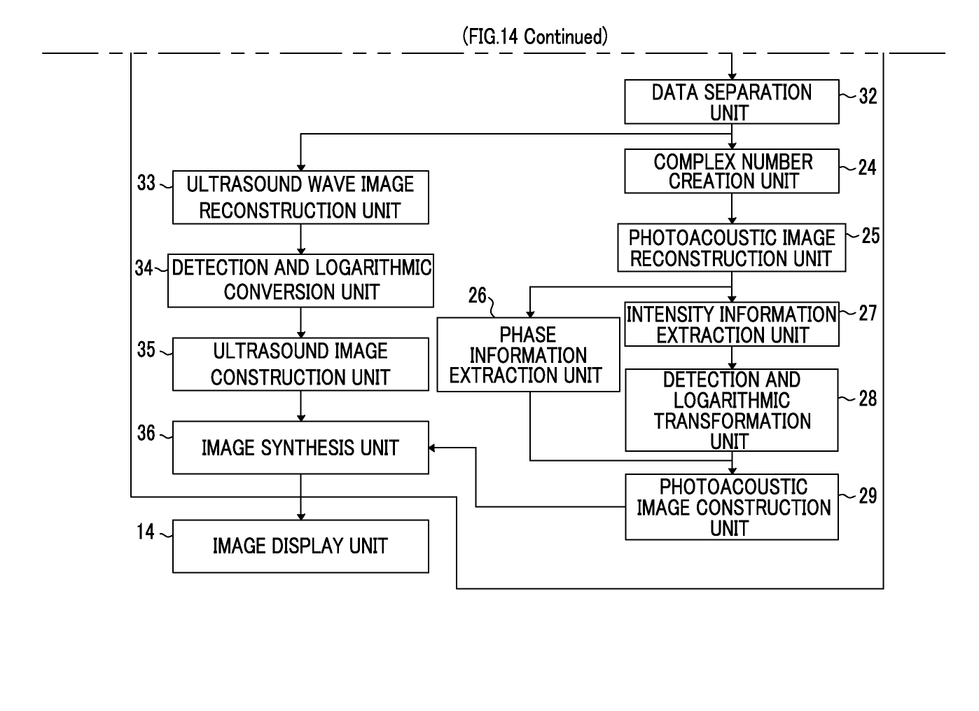

Subsequently, a third embodiment of the invention will be described. FIG. 14 shows a photoacoustic measurement device according to the third embodiment of the invention. In a photoacoustic measurement device 10a of this embodiment, an ultrasound unit 12a has a data separation unit 32, an ultrasound image reconstruction unit 33, a detection and logarithmic conversion unit 34, an ultrasound image construction unit 35, an image synthesis unit 36, and a transmission control circuit 37, in addition to the configuration of the ultrasound unit 12 in the photoacoustic measurement device 10 of the first embodiment shown in FIG. 1. The photoacoustic measurement device 10a of this embodiment is different from the first embodiment in that, in addition to the photoacoustic image, an ultrasound image is generated. The other portions may be the same as those in the first embodiment.

In this embodiment, the probe 11 performs the transmission of an acoustic wave (ultrasonic wave) to the subject and the detection (reception) of a reflected acoustic wave (reflected ultrasonic wave) from the subject for the transmitted ultrasonic wave, in addition to the detection of the photoacoustic signal. At the time of the generation of an ultrasound image, the trigger control circuit 30 sends an ultrasonic transmission trigger signal which instructs the transmission control circuit 37 to perform ultrasonic transmission. If the trigger signal is received, the transmission control circuit 37 causes an ultrasonic wave to be transmitted from the probe 11. After the transmission of the ultrasonic wave, the probe 11 detects a reflected ultrasonic wave from the subject.

The reflected ultrasonic wave detected by the probe 11 is input to the AD conversion unit 22 through the reception circuit 21. The trigger control circuit 30 sends a sampling trigger signal to the AD conversion unit 22 in conformity with the timing of the ultrasonic transmission to start the sampling of the reflected ultrasonic wave. The AD conversion unit 22 stores sampling data (reflected ultrasonic data) of the reflected ultrasonic wave in the reception memory 23.

The data separation unit 32 separates the reflected ultrasonic data stored in the reception memory 23 from the first and second photoacoustic data. The data separation unit 32 transfers the reflected ultrasonic data to the ultrasound image reconstruction unit 33 and transfers the first and second photoacoustic data to the complex number unit 24. The generation of a photoacoustic image based on the first and second photoacoustic data is the same as in the first embodiment. The data separation unit 32 inputs the separated sampling data of the reflected ultrasonic wave to the ultrasound image reconstruction unit 33.

The ultrasound image reconstruction unit 33 generates data of each line of an ultrasound image (reflected acoustic image) based on (the sampling data of) the reflected ultrasonic wave detected by a plurality of ultrasound transducers of the probe 11. For example, the ultrasound image reconstruction unit 33 adds data from 64 ultrasound transducers of the probe 11 with a delay time according to the position of each ultrasound transducer to generate data for one line (delay addition method).

The detection and logarithmic conversion unit 34 obtains an envelope of data of each line output from the ultrasound image reconstruction unit 33 and performs logarithmic conversion for the obtained envelope. The ultrasound image construction unit 35 generates an ultrasound image based on data of each line subjected to logarithmic conversion. The ultrasound image reconstruction unit 33, the detection and logarithmic conversion unit 34, and the ultrasound image construction unit 35 configure an ultrasound image generation unit which generates the ultrasound image based on the reflected ultrasonic wave.

The image synthesis unit 36 synthesizes the photoacoustic image and the ultrasound image. For example, the image synthesis unit 36 performs image synthesis by superimposing the photoacoustic image and the ultrasound image. At this time, it is preferable that the image synthesis unit 36 performs positioning such that the corresponding points are the same positions of the photoacoustic image and the ultrasound image. A synthesized image is displayed on the image display unit 14. Image synthesis may not be performed, and the photoacoustic image and the ultrasound image may be displayed in parallel on the image display unit 14, or the photoacoustic image and the ultrasound image may be switched.

In this embodiment, the photoacoustic measurement device generates the ultrasound image, in addition to the photoacoustic image. A portion which cannot be imaged in the photoacoustic image can be observed by referring to the ultrasound image. The other effects are the same as those in the first embodiment.

In the above-described embodiments, although an example where the first photoacoustic data and the second photoacoustic data are converted to a complex number has been described, the first photoacoustic data and the second photoacoustic data may be separately reconstructed without performing conversion to a complex number. Although the ratio of the first photoacoustic data and the second photoacoustic data is computed using the phase information after conversion to a complex number, even if the ratio is computed from the intensity information of both the first photoacoustic data and the second photoacoustic data, the same effects are obtained. The intensity information can be generated based on signal intensity in a first reconstructed image and signal intensity in a second reconstructed image.

At the time of the generation of the photoacoustic image, the number of wavelengths of pulse laser light irradiated onto the subject is not limited to two, and three beams or more of pulse laser light may be irradiated onto the subject, and a photoacoustic image may be generated based on photoacoustic data corresponding to the respective wavelengths. In this case, the phase information extraction unit 26 may generate the magnitude relationship of relative signal intensity between the photoacoustic data corresponding to the respective wavelengths as phase information. The intensity information extraction unit 27 may generate signal intensity unified from the signal intensities in the photoacoustic data corresponding to the respective wavelengths as intensity information.

In the above-described embodiments, although an example where the first wavelength is 800 nm and the second wavelength is 750 nm has been primarily described, these wavelengths may be in a wavelength band in which laser oscillation is possible, and the invention is not limited to a combination of the wavelength of 800 nm and the wavelength of 750 nm. The second wavelength is not limited to a wavelength at which the laser gain is maximal. For example, when the first wavelength is 800 nm, an arbitrary wavelength between the wavelength of 750 nm, at which the gain is maximal, to the wavelength of 800 nm may be selected as the second wavelength. In this case, a long path filter which transmits light having a wavelength equal to or greater than the wavelength selected as the second wavelength may be inserted on the optical path of the optical resonator such that the laser oscillation wavelength is controlled to the second wavelength.

In the above-described embodiments, an example where the first wavelength is longer than the second wavelength, and the first wavelength and the second wavelength are switched by means of the long path filter when the laser gain coefficient at the first wavelength is lower than the laser gain coefficient at the second wavelength has been described. In contrast, when the first wavelength is shorter than the second wavelength, the first wavelength and the second wavelength can be switched by means of a short path filter (first short path filter) which transmits light having a wavelength equal to or less than the first wavelength. For example, when the first wavelength is 730 nm and the second wavelength is 750 nm, a short path filter which transmits light having a wavelength equal to or less than the wavelength of 730 nm is inserted on the optical path of the optical resonator at the time of the emission of laser light having the wavelength of 730 nm, and the short path filter is removed from the optical path of the optical resonator at the time of the emission of light having the wavelength of 750 nm, whereby laser light having the wavelength of 730 nm and the wavelength of 750 nm can be switched and emitted.

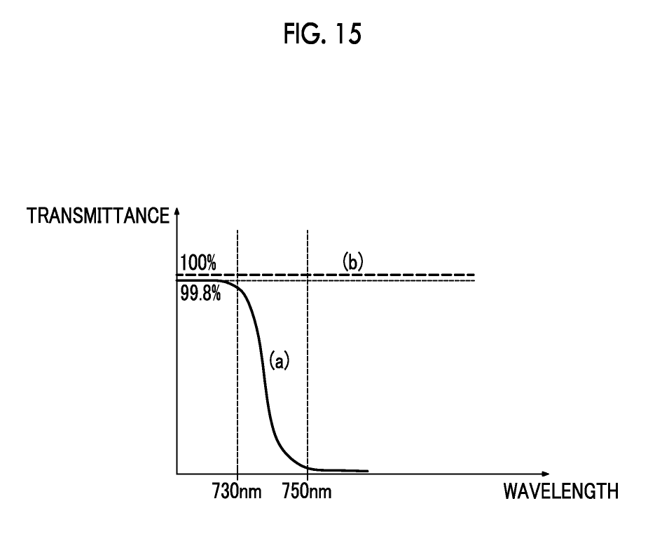

FIG. 15 shows the light transmittance of a wavelength switching unit 56 including a short path filter. In FIG. 15, a graph (a) shows the wavelength characteristic of light transmittance of a short path filter for use in the wavelength switching unit 56 (FIG. 2), and a graph (b) shows the wavelength characteristic of light transmittance at the position of the wavelength switching unit 56 in a state where the wavelength switching unit 56 is removed from the optical path of the optical resonator (FIG. 3B). As shown in the graph (a), the wavelength switching unit (short path filter) 56 transmits light having a wavelength of 700 nm at high light transmittance of, for example, 99.8% and hardly transmits light having a wavelength of 750 nm longer than the wavelength of 700 nm. When the short path filter is removed from the optical path of the optical resonator, since there is no particular member, which blocks light, on the optical path of the optical resonator, light having the wavelength of 730 nm and light having the wavelength of 750 nm are substantially transmitted directly (100%).

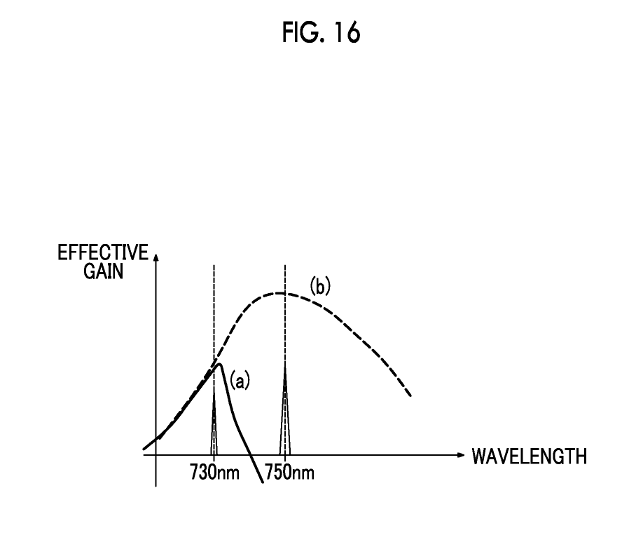

FIG. 16 shows an effective gain of an optical resonator. In FIG. 16, a graph (a) represents an effective gain when the short path filter having the wavelength characteristic shown in the graph (a) of FIG. 15 is inserted on the optical path of the optical resonator, and a graph (b) represents an effective gain when the short path filter is removed. When the short path filter is not inserted (FIG. 3B), as shown in the graph (b) of FIG. 16, the effective gain is maximal around a wavelength of 750 nm as in the wavelength characteristic (FIG. 4) of the laser gain of alexandrite. Laser oscillation occurs at a point (wavelength, excitation power) at which the effective gain >0. When increasing the excitation power, the effective gain is greater than 0 initially at the wavelength of 750 nm at which the effective gain is highest. Accordingly, when the short path filter is not inserted on the optical path of the optical resonator, the optical resonator oscillates at the wavelength of 750 nm of the peak position in the wavelength characteristic of the effective gain.

When a short path filter is inserted on the optical path of the optical resonator (FIG. 3A), since loss in the optical resonator is great on the wavelength side longer than the cutoff wavelength of the short path filter, the effective gain is low, and the effective gain is maximal around the wavelength of 730 nm at which the short path filter transmits light at high light transmittance. Accordingly, when the short path filter is inserted, the optical resonator oscillates at the wavelength of 730 nm of the peak position in the wavelength characteristic of the effective gain.

In the above description, although the short path filter is removed from the optical path of the optical resonator at the time of the emission of light having the second wavelength, alternatively, a configuration may be made in which a short path filter (second short path filter) which transmits light having a wavelength equal to or less than the second wavelength is provided in the wavelength switching unit 56 and the second short path filter is inserted on the optical path of the optical resonator at the time of the emission of light having the second wavelength. The wavelength switching unit 56 may include both a short path filter and a long path filter. For example, the wavelength switching unit 56 includes a short path filter which transmits light having a wavelength equal to or less than a wavelength of 730 nm, a short path filter which transmits light having a wavelength equal to or less than a wavelength of 750 nm or a long path filter which transmits light having a wavelength equal to or greater than a wavelength of 750 nm, and a long path filter which transmits light having a wavelength of 800 nm. In this case, the short path filter or the long path filter is selectively inserted on the optical path of the optical resonator, thereby switching and emitting light having wavelengths of 730 nm, 750 nm, and 800 nm.

In the above-described embodiments, although an alexandrite laser has been primarily described, a laser medium for use in the laser rod 51 (FIG. 2) is not limited to alexandrite. For example, in case of Cr:LiSAF, Cr:LiCAF, or the like, laser oscillation is possible in a wavelength range of 750 nm to 900 nm, and Cr:LiSAF, Cr:LiCAF, or the like may be used in the laser rod 51. In case of Ti:Sapphire, laser oscillation is possible in a wavelength range of 700 nm to 1000 nm, and Ti:Sapphire may be used in the laser rod 51. In FIG. 13, although the dimmer filter 58 which is a dimmer member is disposed in the optical resonator, the invention is not limited thereto, and a configuration may be made in which the dimmer filter 58 is disposed on the optical path of emitted light from the optical resonator.

In the above-described embodiments, although an example where the laser device configures a part of the photoacoustic measurement device has been described, the invention is not limited thereto. The laser device of the invention may be used in a device different from the photoacoustic measurement device. When a laser device does not emit pulse laser light, the Q switch 55 (FIG. 2) may be omitted.

Although the invention has been described based on the preferred embodiments, the laser device and the photoacoustic measurement device of the invention are not limited to the above-described embodiments, and various corrections and alterations may be made from the configuration of the above-described embodiments and still fall within the scope of the invention.

What is claimed is:

1. A laser device which emits light having a plurality of wavelengths including a first wavelength and a second wavelength having a shorter wavelength than the first wavelength, and the second wavelength has a laser gain coefficient higher than a laser gain coefficient at the first wavelength in a laser gain coefficient wavelength characteristic, the laser device comprising:
a laser medium;
an excitation light source which irradiates excitation light onto the laser medium;
an optical resonator which includes a pair of mirrors facing each other with the laser medium interposed therebetween; and
a wavelength switching unit which includes a first long path filter, which transmits light having a wavelength equal to or greater than the first wavelength, and inserts the first long path filter on the optical path of the optical resonator when the wavelength of laser light to be emitted is the first wavelength.

2. The laser device according to claim 1,
wherein the wavelength switching unit transmits both light having the first wavelength and light having the second wavelength when the wavelength of laser light to be emitted is the second wavelength.

3. The laser device according to claim 1,
wherein the wavelength switching unit further includes a second long path filter, which transmits light having a wavelength equal to or greater than the second wavelength, and inserts the second long path filter on the optical path of the optical resonator when the wavelength of laser light to be emitted is the second wavelength.

4. The laser device according to claim 2,
wherein the wavelength switching unit further includes a second long path filter, which transmits light having a wavelength equal to or greater than the second wavelength, and inserts the second long path filter on the optical path of the optical resonator when the wavelength of laser light to be emitted is the second wavelength.

5. The laser device according to claim 3,
wherein the wavelength switching unit has a first region where the first long path filter is disposed and a second region where the second long path filter is disposed, and is configured as a filter rotor which can alternately insert the first region and the second region on the optical path of the optical resonator with rotation displacement.

6. The laser device according to claim 1,
wherein the laser gain coefficient at the second wavelength in the laser gain coefficient wavelength characteristic is maximal, and the wavelength switching unit further includes an optical member, which transmits at least light having the second wavelength, and inserts the optical member on the optical path of the optical resonator when the wavelength of laser light to be emitted is the second wavelength.

7. The laser device according to claim 6,
wherein the wavelength switching unit has a first region where the first long path filter is disposed and a second region where the optical member is disposed, and is configured as a filter rotor which can alternately insert the first region and the second region on the optical path of the optical resonator with the rotation displacement.

8. The laser device according to claim 1,
wherein the laser gain coefficient at the second wavelength in the laser gain coefficient wavelength characteristic is maximal, and the wavelength switching unit removes the first long path filter from the optical path of the optical resonator when the wavelength of laser light to be emitted is the second wavelength.

9. The laser device according to claim 1,
wherein a dimmer member which decreases the amount of transmission of at least light having the second wavelength is inserted on the optical path of the optical resonator or the optical path of emitted light from the optical resonator when the wavelength of laser light to be emitted is the second wavelength.

10. The laser device according to claim 9,
wherein the light transmittance of the dimmer member is selected such that the light intensity of light having the first wavelength output from the laser device and the light intensity of light having the second wavelength are identical.

11. The laser device according to claim 1,
wherein the reflectance of a laser output-side mirror of the pair of mirrors for light having the first wavelength is higher than the reflectance for light having the second wavelength.

12. The laser device according to claim 11,
wherein the reflectance of the laser output-side mirror for light having the first wavelength and the reflectance of the laser output-side mirror for light having the second wavelength are selected such that the effective gain of the optical resonator for the first wavelength and the effective gain of the optical resonator for the second wavelength are identical.

13. The laser device according to claim 9,
wherein the input energy of excitation light to the laser medium is the same between when the wavelength of laser light to be emitted is the first wavelength and when the wavelength of laser light to be emitted is the second wavelength.

14. The laser device according to claim 1, further comprising:
a Q switch which is disposed on the optical path of the optical resonator.

15. A photoacoustic measurement device comprising:
the laser device according to claim 1 which emits light having a plurality of wavelengths including a first wavelength and a second wavelength having a wavelength shorter than the first wavelength, and the second wavelength has a laser gain coefficient higher than a laser gain coefficient at the first wavelength in a laser gain coefficient wavelength characteristic, the laser device having a laser medium, an excitation light source which irradiates excitation light onto the laser medium, an optical resonator which includes a pair of mirrors facing each other with the laser medium interposed therebetween, and a wavelength switching unit which includes a first long path filter, which transmits light having a wavelength equal to or greater than the first wavelength, and inserts the first long path filter on the optical path of the optical resonator when the wavelength of laser light to be emitted is the first wavelength;
a detection unit which detects a photoacoustic signal generated in a subject when laser light having the first wavelength and the second wavelength is irradiated onto the subject and generates first photoacoustic data and second photoacoustic data corresponding to the first wavelength and the second wavelength; and
an intensity ratio extraction unit which extracts the magnitude relationship of relative signal intensity between the first photoacoustic data and the second photoacoustic data.

16. The photoacoustic measurement device according to claim 15, further comprising:
a photoacoustic image construction unit which generates a photoacoustic image based on the first photoacoustic data and the second photoacoustic data.

17. The photoacoustic measurement device according to claim 16, further comprising:
an intensity information extraction unit which generates intensity information representing signal intensity based on the first photoacoustic data and the second photoacoustic data,
wherein the photoacoustic image construction unit determines the gradation value of each pixel of the photoacoustic image based on the intensity information and determines the display color of each pixel based on the extracted magnitude relationship.

18. The photoacoustic measurement device according to claim 17, further comprising:
a complex number unit which generates complex data, in which one of the first photoacoustic data and the second photoacoustic data is a real part and the other data is an imaginary part; and
a photoacoustic image reconstruction unit which generates a reconstructed image from the complex data by a Fourier transformation method,
wherein the intensity ratio extraction unit extracts phase information as the magnitude relationship from the reconstructed image, and the intensity information extraction unit extracts the intensity information from the reconstructed image.

19. A laser device which emits light having a plurality of wavelengths including a first wavelength and a second wavelength having a longer wavelength than the first wavelength, and the second wavelength has a laser gain coefficient higher than a laser gain coefficient at the first wavelength in a laser gain coefficient wavelength characteristic, the laser device comprising:
a laser medium;
an excitation light source which irradiates excitation light onto the laser medium;
an optical resonator which includes a pair of mirrors facing each other with the laser medium interposed therebetween; and
a wavelength switching unit which includes a first short path filter, which transmits light having a wavelength equal to or less than the first wavelength, and inserts the first short path filter on the optical path of the optical resonator when the wavelength of laser light to be emitted is the first wavelength.

20. The laser device according to claim 19,
wherein the wavelength switching unit further includes a second short path filter, which transmits light having a wavelength equal to or less than the second wavelength, and inserts the second short path filter on the optical path of the optical resonator when the wavelength of laser light to be emitted is the second wavelength.
